(19) United States
(12) Patent Application Publication
(10) Pub. No.: US 2024/0115499 A1
(43) Pub. Date: Apr. 11, 2024

(54) DHA and Choline Emulsion

(71) Applicant: Capsugel Belgium NV, Bornem (BE)

(72) Inventors: Tyler Oswald White, Greenwood, SC (US); Kelli Fowler, Greenwood, SC (US)

(21) Appl. No.: 17/769,038

(22) PCT Filed: Oct. 15, 2020
(86) PCT No.: PCT/US2020/055720

§ 371 Date: Apr. 14, 2022

Related U.S. Application Data
(60) Provisional application No. 62/915,276, filed on Oct. 15, 2019.

(51) Int. Cl. A61K 9/107; A61K 9/48; A61K 31/14

(52) U.S. Cl. CPC A61K 9/107; A61K 9/4808; A61K 9/4858; A61K 9/4866; A61K 31/14

(57) ABSTRACT

The present disclosure is directed to an oil-in-water emulsion containing at least one hygroscopic ingredient and an edible oil. The emulsion has a water activity of less than about 0.6. The emulsion may be incorporated into an oral dosage form, including a capsule oral dosage form. The ingredients provided in the emulsion are capable of improving the health or well-being of a human or animal.

### CROSS REFERENCE TO RELATED APPLICATIONS

This application claims priority to and the benefit of U.S. Provisional Application No. 62/915,276, filed Oct. 15, 2019, which is expressly incorporated herein by reference in its entirety.

### BACKGROUND

Many compositions, e.g. pharmaceuticals or nutraceuticals, may need to be formulated as emulsions, generally due to one or more necessary components being substantially water-insoluble. However, formulating certain products in emulsion form raises its own problems, for example, the stability of the emulsion itself and of its components, and the increased volume of an emulsion relative to a concentrated, solid formulation.

Furthermore, there is a need for an emulsion that can be formulated and incorporated into a suitable capsule for oral delivery. Currently many pharmaceutical or nutraceutical emulsions are not suitable for formulation with certain capsules. Indeed, many nutritional emulsions, when incorporated into certain capsules, may degrade or disintegrate the capsule.

Accordingly, there exists a need for producing an emulsion that has enhanced stability that can be formulated into capsule form.

### SUMMARY

The present disclosure concerns an oil-in-water emulsion containing at least one hygroscopic ingredient, at least one edible oil, water, and at least one functional gum, wherein the emulsion has a water activity (aw) of less than about 0.6. In certain embodiments, the hygroscopic ingredient can include choline. In certain embodiments, the at least one edible oil includes a docosahexaenoic acid (DHA)-containing oil. The emulsion may additionally contain one or more emulsifiers, stabilizers, water-soluble ingredients, and/or fat-soluble ingredients.

The present disclosure also provides an oral dosage form that includes a capsule containing the emulsion described according to the example embodiments provided herein. The capsule may include a suitable hydroxypropyl methylcellulose (HPMC)-based capsule. In certain embodiments, the capsule may include a modified HPMC-based capsule.

The emulsions disclosed herein may be incorporated into any suitable nutritional composition or supplement and can be used to improve the overall health and wellness of a subject, such as a human. The methods provided herein may include administering a nutritional supplement containing the emulsion provided herein to the subject. The emulsion can be administered via any suitable oral dosage form, such as a capsule. Further, the emulsions disclosed herein may be incorporated into a suitable composition or product and can be used as a prenatal and/or multivitamin/mineral supplement.

### DETAILED DESCRIPTION

It is to be understood by one of ordinary skill in the art that the present discussion is a description of exemplary embodiments only and is not intended as limiting the broader aspects of the present disclosure.

The term “therapeutically effective amount” as used herein, with respect to the composition described, shall mean that dosage, or amount of composition, that provides the specific pharmacological or nutritional response for which the composition is administered or delivered to subjects in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation.

The term “delivering” or “administering” as used herein, refers to any route for providing the composition, product, or a nutraceutical, to a subject as accepted as standard by the medical community. For example, the present invention contemplates routes of delivering or administering that include oral ingestion plus any other suitable route of delivery including transdermal, intravenous, intraperitoneal, intramuscular, pulmonary, topical and subcutaneous. Additionally, technology such as nanotechnology, micronization, and spray-dried dispersions may be used to enhance the mode of delivery or administration.

As used herein, the term “mammal” can include without limitation canine, equine, feline, bovine, ovine, human, or porcine mammals.

As used herein, the term “oil-in-water emulsion” can include an emulsion having oil, such as oil droplets, suspended in an aqueous phase.

All percentages, parts and ratios as used herein are by weight of the emulsion, unless otherwise specified. All such weights as they pertain to listed ingredients are based on the active level and, therefore, do not include solvents or by-products that may be included in commercially available materials; unless otherwise specified.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be counted as supporting a range of from 2 to 8, from 3 to 7, 5, 6, from 1 to 9, from 3.6 to 4.6, from 3.5 to 9.9, and so forth.

All references to singular characteristics or limitations of the present invention shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The embodiments of the present invention can comprise, consist of or consist essentially of the essential elements and limitations of the invention described herein, as well as any additional or optional ingredients, components, or limitations described herein or otherwise useful in nutrition formula applications.

Other features and aspects of the present disclosure are discussed in greater detail below.

In general, the present disclosure is directed to emulsions, particularly oil-in-water emulsions that contain at least one hygroscopic ingredient, at least one edible oil, water, and at least one functional gum, wherein the emulsion has a water activity (aw) of less than about 0.6. In certain embodiments, the hygroscopic ingredient can include choline. In certain embodiments, the at least one edible oil includes a DHA-containing oil. The emulsions provided herein may be incorporated into products or compositions, such as nutritional supplements, medicinal and/or food formulations, or beverages for administering to humans and animals that can be used as a daily or periodic nutritional supplement. Of particular advantage is that the composition can be formulated to provide multiple health benefits simultaneously.

Formulating nutritional emulsions, particularly those containing two immiscible ingredients, such as a hygroscopic ingredient in combination with an edible oil, such as algal oils containing DHA, can be particularly difficult. For example, mixtures of water with hygroscopic ingredients and algal oils often times produce immiscible mixtures and unstable physical emulsions. Thus, formulating a nutritional supplement containing an emulsion of both a hygroscopic ingredient in water and edible oils is difficult if not impossible. Consumers desiring to supplement their diet with certain ingredients, such as choline and DHA, must often take two or more separate oral dosage forms in order to achieve their required dietary supplementation of these ingredients. The increased number of oral dosage forms can lead to decreased compliance from consumers since many consumers may forget to take additional oral dosage form supplements. Accordingly, when certain nutrients require multiple oral dosage forms, consumer compliance can be low, thus resulting in poor nutritional supplementation.

In one embodiment, for instance, the composition of the present disclosure includes an oil-in-water emulsion containing at least one hygroscopic ingredient and at least one edible oil. The emulsion has a water activity of less than about 0.6. The emulsion may be formulated to provide daily nutritional supplementation to a subject, such as a mammal, such as a human.

In some embodiments, the oil-in-water emulsion may contain one or more hygroscopic ingredients. For example, in certain embodiments the hygroscopic ingredient can include choline, carnitine, betaine, amino acids and amino acid derivatives (i.e. L-tyrosine, L-cysteine HCl, L-taurine, L-glutamic acid, N-acetyl-L-tyrosine, alpha ketoglutaric acid, L-Arginine HCl, I-ornithine HCl, L-glutamine, L-alanine, L-proline) L-alpha glycerylphosphorylcholine (Alpha GPC), sorbitol, mannitol, maltitol, xylitol, and combinations thereof.

Additional hygroscopic ingredients may be added to the oil-in-water emulsion based on the intended end use for the nutritional supplement.

As noted, one suitable hygroscopic ingredient includes choline. In general, choline is a nutrient that is naturally present in some foods. Choline is a source of methyl groups needed for many steps in metabolism. For example, the body utilizes choline to synthesize phosphatidylcholine and sphingomyelin, which are two major phospholipids vital for cell membranes. In addition, choline is needed to produce acetylcholine, which is an important neurotransmitter for memory, mood, muscle control, and other brain and nervous system functions. Choline may also play an important role in modulating gene expression, cell membrane signaling, lipid transport, metabolism, and early brain development.

Humans are capable of producing choline endogenously in the liver, mostly as phosphatidylcholine, however the amount that the body naturally synthesizes is not sufficient to meet human needs. Accordingly, humans need to obtain additional choline from their diet. However, most people in the United States consume less than the Adequate Intake (AI) for choline. Choline deficiency can cause muscle damage, liver damage, and nonalcoholic fatty liver disease. Furthermore, certain groups of individuals are at an increased risk of choline inadequacy. One group is pregnant women. For example, approximately 90-95% of pregnant women consume less choline than the AI. However, many prenatal supplements contain little if any choline. This may be due to formulation difficulties associated with incorporating choline into prenatal supplements that contain other necessary ingredients, such as DHA. Other groups at risk for choline inadequacy include people with certain genetic alterations, such as those having altered genes involved in the metabolism of choline, folate, and methionine, and patients requiring total parenteral nutrition. Accordingly, there is a need for nutritional supplements, especially prenatal nutritional supplements, that contain an adequate amount of choline to ensure nutritional supplementation.

The term “choline” refers to quaternary ammonium salts containing the N,N,N-trimethylethanolammonium cation and having the structure shown below:

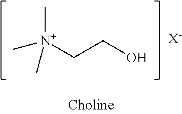

In one preferred embodiment, counterion X⁻ is selected from chloride, hydroxide, citrate, bitartrate and mixtures thereof.

Unless stated otherwise, within the context of the present disclosure, the term “choline” should be intended to identify all the choline present in the nutritional supplements of the disclosure, in free form or as a salt thereof.

Choline may be incorporated in the emulsion of the present disclosure as such or in the form of one physiologically acceptable salt such as, for example, choline chloride, choline citrate, choline bitartrate, or mixtures thereof.

In some embodiments, the oil-in-water emulsion contains from about 30% to about 60% by weight of choline. In some embodiments, the oil-in-water emulsion contains from about 35% to about 55% by weight of choline. In some embodiments, the oil-in-water emulsion contains from about 40% to about 50% by weight of choline.

In certain embodiments, where one or more hygroscopic ingredients are included in the oil-in-water emulsion, the oil-in-water emulsion may contain from about 30% to about 60% of total hygroscopic ingredients. For example, in certain embodiments if the oil-in-water emulsion contains about 40% of choline, then the oil-in-water emulsion may contain up to 20% by weight of one or more additional hygroscopic ingredients, such as up to 15% by weight, such as up to 10% by weight, such as up to 5% by weight. Thus, in certain embodiments, the oil-in-water emulsion may contain from about 0% by weight to about 60% by weight of one or more hygroscopic ingredients in addition to choline. In certain embodiments, the oil-in-water emulsion may contain from about 5% by weight to about 55% by weight of one or more hygroscopic ingredients in addition to choline. In certain embodiments, the oil-in-water emulsion may contain from about 10% by weight to about 50% by weight of one or more hygroscopic ingredients in addition to choline. In certain embodiments, the oil-in-water emulsion may contain from about 15% by weight to about 45% by weight of one or more hygroscopic ingredients in addition to choline. In certain embodiments, the oil-in-water emulsion may contain from about 20% by weight to about 40% by weight of one or more hygroscopic ingredients in addition to choline. In certain embodiments, the oil-in-water emulsion may contain from about 25% by weight to about 35% by weight of one or more hygroscopic ingredients in addition to choline.

The emulsion may also include an edible oil, such as an algal oil containing DHA. DHA is an omega-3 fatty acid mainly found in seafood, such as fish, shellfish, fish oils, and algal oils. DHA is a structural component of skin cells, eye cells, and brain cells. DHA is a carboxylic acid with a 22-carbon chain having six cis double bonds. DHA is the most abundant omega-3 fatty acid present in the brain and retina. For example, it is estimated that DHA comprise 40% of the polyunsaturated fatty acids in the brain at 60% of the polyunsaturated fatty acids in the retina. DHA modulates the carrier-mediated transport of choline, glycine, and taurine. DHA deficiency may be associated with cognitive decline. Furthermore, the use of DHA in food or dietary supplements may reduce risk of hypertension and coronary heart disease.

While DHA can be synthesized by the body from alpha-linolenic acid (ALA), this process is very inefficient. In fact, it is estimated that only about 0.1% to about 0.5% of ALA is converted to DHA by the body. Accordingly, because DHA cannot be synthesized by the body in significant amounts, it must be supplied by the diet or by dietary supplementation.

Accordingly, in certain embodiments the emulsion contains an edible oil containing DHA. The edible oil may be incorporated into an oil-in-water emulsion. The oil-in-water emulsion may contain from about 30% to about 60% by weight of an edible oil containing DHA. In certain embodiments, the oil-in-water emulsion may contain from about 40% to about 50% by weight of an edible oil containing DHA.

In certain embodiments, the edible oil may include an oil derived from an marine source, such as an algal oil or a fish oil. In some embodiments, the edible oil comprises Algarithum® DHA 530.

In certain embodiments, the edible oil may include one or more of the following: algal oil, hemp oils or extracts, edible seed oils such as sunflower seed oil, tomato seed oil, safflower seed oil, raspberry seed oil, flax seed oil, borage seed oil, fish oils, ahiflower oil, grapeseed oil, sea buckthorn oil, evening primrose oil, argan oil, castor oil, black cumin oil, rice bran oil, ylang ylang oil, oil of oregano, perilla seed oil, wheat germ oil, black currant oil, chia seed oil, inca inchi seed oil, extra virgin olive oil, palm oil, soybean oil, MCT oil, coconut oil, and combinations thereof.

In some embodiments, one or more edible oils may be incorporated into the oil-in-water emulsion in an amount of from about 10% to about 40% by weight. In some embodiments, one or more edible oils may be incorporated into the oil-in-water emulsion in an amount of from about 15% to about 35% by weight. In some embodiments, one or more edible oils may be incorporated into the oil-in-water emulsion in an amount of from about 20% to about 30% by weight. In some embodiments, the one or more edible oils may be incorporated into the oil-in-water emulsion in an amount equal to or less than 40% by weight, such as less than 35% by weight, such as less than 30% by weight, such as less than 25% by weight, such as less than 20% by weight, such as less than 15% by weight, such as less than 10% by weight, such as less than 10% by weight, such as less than 5% by weight.

One or more additional edible oils may be added to the oil-in-water emulsion based on the intended end use for the nutritional supplement.

The oil-in-water emulsion may also contain at least one functional gum, such as gum arabic. Gum arabic, in general, is a complex mixture of glycoproteins and polysaccharides, including arabinose and galactose. Gum arabic is generally soluble in water and is edible. In some embodiments, the gum arabic may be comprised of a 100% modified gum arabic, such as Ticamulsion® A-2010 gum arabic powder. In certain embodiments, the gum arabic may be a mixture or blend of gum arabic and modified gum arabic. For example, in certain embodiments, the gum arabic may comprise Ticamulsion® 3020.

In certain embodiments, the oil-in-water emulsion contains from about 10% to about 30% by weight of gum arabic. In some embodiments, the oil-in-water emulsion contains from about 15% to about 25% by weight of gum arabic. In some embodiments, the oil-in-water emulsion contains less than about 20% by weight of gum arabic, such as less than 15%, such as less than 10%, such at less than 5%.

The oil-in-water emulsion may also contain water. In certain embodiments, the oil-in-water emulsion contains deionized water. Still, in certain embodiments, the oil-in-water emulsion may contain any water suitable for human ingestion and incorporation into dietary supplements designed for human ingestion.

The amount of water incorporated into the oil-in-water emulsion can vary depending on the desired hygroscopic and water-soluble ingredients that are incorporated into the oil-in-water emulsion. In certain embodiments, the oil-in-water emulsion may contain from about 5% to 35% by weight of water. In some embodiments, the oil-in-water emulsion may contain from about 10% to about 30% by weight of water. In some embodiments, the oil-in-water emulsion may contain from about 15% to about 20% by weight of water. In some embodiments, the oil-in-water emulsion may contain less than about 20% by weight of water, such as less than about 15% by weight of water, such as less than about 10% by weight of water.

In certain embodiments, the oil-in-water emulsions may have a water activity of less than about 0.6, such as less than about 0.55, such as less than about 0.5, such as less than about 0.45, such as less than about 0.40, such as less than about 0.35, such as less than about 0.30, such as less than about 0.25, such as less than about 0.20, such as less than about 0.15, such as less than about 0.10. Generally, water activity (a_{w}) described the equilibrium amount of water available for hydration of materials. Water activity generally measures the partial vapor pressure of water in the solution and divides that value by the partial vapor pressure of water at the same temperature to give a final reading. Water activity (a_{w}) can be measured according to the following:

a_{w}=p/p*

Without being bound by any particular theory, it was discovered herein that emulsions having water activities over 0.6 may lead to physical instability of the emulsions. Furthermore, it was discovered herein that emulsions having a water activity greater than 0.6 may cause disintegration or dissolve certain HPMC capsules.

In some embodiments, the oil-in-water emulsion may contain one or more stabilizers or suspension promoting agents. For example, in certain embodiments, the oil-in-water emulsion may contain one or more gum, such as gellan gum or xanthum gum. If included, the gellan gum or xanthum gum may be present in an amount of less than about 3.5% by weight of the oil-in-water emulsion, such as less than about 2.5% by weight, such as less than about 1.5% by weight, such as less than about 1.0% by weight, such as less than about 1.0% by weight.

In other embodiments, the oil-in-water emulsion may contain one or more stabilizers such as silica. If included, silica may be present in an amount of less than about 2% by weight, such as less than about 1.5% by weight, such as less than about 1% by weight, such as less than about 0.5% by weight.

In some embodiments, the oil-in-water emulsion may also contain one or more water-soluble ingredients or nutrients. In certain embodiments, the one or more water-soluble ingredients or nutrients may be incorporated into the water phase of the oil-in-water phase emulsion. Suitable water soluble ingredients that may be incorporated include, but are not limited to, ascorbic acid, gamma-aminobutyric acid (GABA), sodium chloride, thiamine hydrochloride, riboflavin-5-phosphate sodium, nicotinic acid, folic acid, cyanocobalamin, methylcobalamin, biotin, sodium ascorbate, citicoline, caffeine anhydrous, yohimbine HCl, nicotinamide adenine dinucleotide (NADH), pyridoxine HCl, and combinations thereof.

In certain embodiments, at least one or more water-soluble ingredients may be incorporated in the oil-in-water emulsion in an amount of from about 0% by weight to about 50% by weight. For example, in some embodiments, the oil-in-water emulsion contains less than about 50% by weight of one or more water-soluble ingredients, such as less than about 40% by weight, such as less than about 30% by weight, such as less than about 20% by weight, such as less than about 10% by weight, such a less than about 5% by weight.

Additional water-soluble ingredients may be added to the oil-in-water emulsion based on the intended end use for the nutritional supplement.

In some embodiments, the oil-in-water emulsion may also contain one or more fat-soluble ingredients or nutrients. In certain embodiments, the one or more fat-soluble ingredients or nutrients may be incorporated into the oil phase of the oil-in-water phase emulsion. Suitable fat-soluble ingredients include, but are not limited to retinol, vitamin E sourced from mixed tocopherols, beta carotene, ubiquinone, lecithin, sunflower lecithin, vitamin D, cannabinoids, hemp extracts, vitamin K, phosphatidyl choline, and combinations thereof.

In certain embodiments, at least one or more fat-soluble ingredients may be incorporated in the oil-in-water emulsion in an amount of from about 0% by weight to about 50% by weight. For example, in some embodiments, the oil-in-water emulsion contains less than about 50% by weight of one or more fat-soluble ingredients, such as less than about 40% by weight, such as less than about 30% by weight, such as less than about 20% by weight, such as less than about 10% by weight, such a less than about 5% by weight.

Additional fat-soluble ingredients may be added to the oil-in-water emulsion based on the intended end use for the nutritional supplement.

The oil-in-water emulsion of the present disclosure may also comprise an antioxidant in addition to the choline and edible oil source. Without being bound by any particular theory, t is believed that the addition of an antioxidant may prevent or reduce oxidation of the ingredients contained in the oil-in-water emulsion, such as an algal oil containing DHA, and may be further beneficial to the consumers nutritional health. Examples of the antioxidant that may be added to the composition or product include vitamins, stilbenoids, curcumininoids, tannins, flavones, flavonols, flavan-3-ols, flavanones, anthocyanidins, anthocyanins, isoflavones, flavanonols, proanthocyanidins, dihydroxybenzoic acids, carotenoids, and pyridine alkaloids.

For example, in one embodiment the antioxidant comprises one or more antioxidant vitamins, which are desirably selected from the group consisting of vitamin B, vitamin C, and vitamin E and desirably is derived from a food or plant source, for example, green tea, acai, and the like.

In one embodiment, the antioxidant comprises an extract containing one or more stilbenoids, which are desirably selected from the group consisting of resveratrol, piceatannol, pterostilbene, and astringin. In one embodiment the antioxidant comprises one or more curcumininoids, which are desirably selected from the group consisting of curcumin, demethoxycurcumin, and bisdemethoxycurcumin.

In one embodiment, the antioxidant comprises one or more tannins selected from the group consisting of hydrolyzable tannins, gallic acid, gallic acid C1-12 alkyl esters, ethyl gallate, propyl gallate, octyl gallate, dodecyl gallate, theaflavin esters of gallic acid, and condensed tannins (e.g., proanthocyanidins, prodelphinidins, procyanidins, oligomeric proanthocyanidins, leukocyanidins, leucoanthocyanins).

In one embodiment, the antioxidant comprises one or more flavones selected from the group consisting of apigenin, luteolin, tangeritin, chrysin, 6-hydroxyflavone, baicalein, scutellarein, wogonin, and orientin.

In one embodiment, the antioxidant comprises one or more flavonols selected from the group consisting of 3-hydroxyflavone, azaleatin, fisetin, galangin, gossypetin, isorhamnetin, kaempferide, kaempferol, morin, myricetin, natsudaidain, pachypodol, quercetin, rhamnazin, and rhamnetin.

In one embodiment, the antioxidant comprises one or more flavan-3-ols selected from the group consisting of catechin, epicatechin, gallocatechin, epigallocatechin, catechin gallate, epicatechin gallate, epigallocatechin gallate, epiafzelechin, fisetinidol, guibourtinidol, mesquitol, robinetinidol.

In one embodiment, the antioxidant comprises one or more flavanones selected from the group consisting of butin, eriodictyol, hesperetin, hesperidin, homoeriodictyol, isosakuranetin, naringenin, pinocembrin, poncirin, sakuranetin, sakuranin, and sterubin.

In one embodiment, the antioxidant comprises one or more anthocyanins selected from the group consisting of aurantinidin, cyaniding, delphinidin, europinidin, luteolinidin, pelargonidin, malvidin, peonidin, petunidin, rosinidin.

In one embodiment, the antioxidant comprises one or more anthocyanins selected from the group consisting of aurantinidin, cyaniding, delphinidin, europinidin, luteolinidin, pelargonidin, malvidin, peonidin, petunidin, rosinidin.

In one embodiment, the antioxidant comprises one or more pyridine alkaloids selected from the group consisting of trigonelline, arecoline, ricinine, actinidine, gentianine, and gentialutine.

In one embodiment, the antioxidant comprises one or more carotenoids selected from the group consisting of lutein, lycopene, and beta-carotene.

In some embodiments, the antioxidant comprises one or more extracts selected from alfalfa (Medicago sativa), green tea (Camellia sinesis), turmeric (Curcuma longa), edelweiss, eucommia (Eucommia ulmoides), white tea, black tea, grape (Vitis vinifera) seed, acai fruit (Euterpe oleracea), cocoa (Theobroma cacao), milk thistle, soy, thyme, jiaogulan (Gynostemma pentaphyllum)(rich in saponins, e.g., gypenosides), rooibos, Chamomilla Recutita, Scutellaria Baicalensis Root, carrot, kiwi (Actinidia chinensis), Ampelopsis Grossedentata, raspberry seed (Rubus idaeus), pomegranate seed, elderberry seed, grapefruit seed, black currant seed, cranberry seed (Vaccinium macrocarpon), bilberry (vaccinium myrtillus) seed, Pueraria Lobata Root, Gingko Biloba, Madagascar ambiatry (Vernonia appendiculata), Burdock (Arctium lappa) root, sage, myrtle, hibiscus, Panax ginseng root, olive (Olea Europaea) leaf, sweet pea, rosemary, sage, oregano, thyme, black pepper, clove, cinnamon (Cinnamomum cassia Presl), coriander, Chinese prickly ash, lemon grass, star anise, mint, sweet basil, bay leaf (Laurus nobilis L), and pine bark (Pinus pinaster) extracts, more preferably eucommia (Eucommia ulmoides), green tea (Camellia sinesis), grape (Vitis vinifera) seed, acai fruit (Euterpe oleracea), turmeric (Curcuma longa), and cocoa (Theobroma cacao) extracts.

In certain embodiments, the oil-in-water emulsion may contain a fat-soluble antioxidant, such as Vitamin E or rosemary extract. In certain embodiments where a fat-soluble antioxidant is desired, the fat-soluble antioxidant may be added to oil-in-water dispersion and dispersed in the oil phase of the oil-in-water dispersion.

In certain embodiments, the oil-in-water emulsion may contain a water-soluble antioxidant, such as vitamin C. In certain embodiments where a water-soluble antioxidant is desired, the water-soluble antioxidant may be added to oil-in-water dispersion and dispersed in the aqueous phase of the oil-in-water dispersion.

Disclosed herein are example methods for formulating the oil-in-water emulsions disclosed herein. These methods are provided by way of example only. In general, any suitable method for forming an oil-in-water emulsion may be used to formulate the oil-in-water emulsions disclosed herein.

In some embodiments, the oil-in-water emulsions disclosed herein may be formed by mixing water with at least one stabilizer, such as gellan gum and shearing the mixture. The mixture of water and at least one stabilizer can be heated to a temperature of up to 80° C. Choline can then be added to the sheared mixture of water and gellan gum and stirred until a homogenous mixture is formed. The mixture can then be cooled to room temperature. At least one functional gum, such as gum arabic, can then be added to the mixture. At least one edible oil, such as an algal oil containing DHA, can then be added to the mixture. The mixture is then sheared until a homogenous, oil-in-water emulsion is formed. Additional ingredients, such as silica, may then be added to the oil-in-water emulsion and stirred. In some embodiments, additional ingredients such as additional water-soluble ingredients, fat soluble ingredients, hygroscopic ingredients, or other edible oils may be added to the mixture and stirred until a homogenous, oil-in-water emulsion is formed.

In some embodiments, the oil-in-water emulsions disclosed herein may be formed by mixing water with choline, such as choline chloride, and heating the mixture to a temperature of 80° C. while stirring the mixture. At least one stabilizer, such as gellan gum, including both high acyl gellan gum and low acyl gellan gum, can then be added to the mixture and the mixture can then be sheared. If the temperature of the mixture has decreased during the shearing process, then the mixture can be re-heated to a temperature of about 80° C. and held at this temperature for a time period of about 10 minutes. The mixture can then be cooled to room temperature. Gum arabic and an edible oil containing DHA can then be added to the mixture and the entire mixture is then sheared until the mixture forms a homogenous, oil-in-water emulsion. If desired, silica may be added to the oil-in-water emulsion and stirred until granules of silica dissolve into the oil-in-water emulsion. In certain embodiments, where silica is added, the emulsion may be stirred for a period of from about 12 to 18 hours.

In certain embodiments, little to no heat is applied to the mixture or emulsion once the functional gum, such as gum arabic is added to the solution. For example, subjecting the mixture to heat after addition of the gum arabic can cause the solution to separate thus not forming a physically stable oil-in-water emulsion. Additional gums that may be optionally added to the oil-in-water emulsion include, but are not limited to, arabinogalactan, persian gum, guar gum, propylene glycol alginate, and combinations thereof.

In some embodiments, the oil-in-water emulsion disclosed herein may be provided in any suitable dosage form, such as capsules, tablets, gummy chewables, edible films, lozenges, powders, liquid suspensions, syrups, lipid micelles, spray-dried dispersions, nanoparticles, and the like. In one embodiment, the dosage form is an oral dosage form such as a capsule. Capsules are well-known dosage forms that normally consist of a shell filled with one or more specific substances, i.e. the oil-in-water emulsion disclosed herein. The capsule shell may be a soft shell or a hard shell containing film-forming polymers, such as gelatin, modified starches, modified cellulosed, etc.

In certain embodiments, the oil-in-water emulsion may be formulated to be provided in a capsule. In certain embodiments, the capsule may include any suitable HPMC-based capsule. Many HPMC capsules deteriorate or easily disintegrate when oil-in-water emulsions are added to the capsule. This is largely due to contact between the HPMC-based capsule and the aqueous phase of the oil-in-water emulsion. Exposure of the HPMC-base capsule to the aqueous phase of an oil-in-water emulsion causes the capsule to deteriorate and/or disintegrate. Surprisingly, however, the inventors herein discovered that the oil-in-water emulsions disclosed herein are suitable for formulation into HPMC-based capsules and furthermore, no capsule disintegration was observed. Advantageously, the oral dosage form provided herein may include an HPMC-based capsule containing the oil-in-water emulsion disclosed herein.

In certain embodiments, the HPMC-base capsule includes capsules manufactured by Lonza® Inc. under the tradename of Vcaps®, Vcaps® Plus, or DRcaps®. In some embodiments, the capsule may include a modified HPMC-capsule that contains gellan gum and/or thalate. In certain embodiments, the capsule may include a HPMC capsule that has been treated so as to be acid resistant. For example, the capsule can include an enteric capsule containing hydroxypropyl methylcellulose acetate succinate (HPMCAS).

In certain embodiments, the capsule may contain a certain fill weight range or fill volume range. For example, in certain embodiments, the capsule may be filled with from about 150 mg to about 1050 mg of the oil-in-water emulsion. In some embodiments, the capsule may be filled with about 150 mg of the oil-in-water emulsion. In some embodiments, the capsule may be filled with about 20 mg of the oil-in-water emulsion. In some embodiments, the capsule may be filled with about 280 mg of the oil-in-water emulsion. In some embodiments, the capsule may be filled with about 380 mg of the oil-in-water emulsion. In some embodiments, the capsule may be filled with about 500 mg of the oil-in-water emulsion. In some embodiments, the capsule may be filled with about 700 mg of the oil-in-water emulsion. In certain embodiments, the capsule may be filled with about 1,050 mg of the oil-in-water emulsion.

Alternatively, the oil-in-water emulsion may be contained in a nutritional product, such as a food product or in a beverage. For example, in certain embodiments, the oil-in-water emulsion may be incorporated into a liquid nutritional product, such as a nutritional supplement or infant formula, to be consumed by a human. Furthermore, the oil-in-water emulsions provided herein may be formulated into other nutritional products that are suitable for administration to animals, such as cattle, horses, goats, sheep, rabbits, dogs, cats, hamsters, etc. The oil-in-water emulsions provided herein may be added to any liquid nutritional product designed to provide nutritional supplementation to a mammal.

As described above, in one embodiment, the oil-in-water emulsion of the present disclosure can be formulated as a nutritional supplement or product that is taken orally. It should be understood, however, that the composition or product of the present disclosure can be administered to a human or animal using any suitable method. For example, in certain embodiments, the oil-in-water emulsion can be incorporated into a topical composition that is intended to be applied to the skin of a user. When formulated as a topical composition, for instance, the oil-in-water emulsion can be further formulated with solvents, surfactants, emulsifiers, consistency factors, conditioners, emollients, skin care ingredients, moisturizers, thickeners, lubricants, preservatives, and various different dermatological ingredients. When applied to the skin of a user, the oil-in-water emulsion can be used to improve skin health and/or can be formulated so as to be absorbed into the body.

In addition to being directed to a composition or a nutritional supplement, the present disclosure is also directed to a method for administering a nutritional supplement containing an oil-in-water emulsion to a human or animal. The nutritional supplement is administered to the mammal in a therapeutically effective amount so as to provide nutritional health benefits. In general, as described above, the supplement of the present disclosure can be administered orally. In other embodiments, however, the supplement can be administered subcutaneously or via a patch.

### Examples

Table 1, illustrated below, provides an example embodiment of the profile of an oil-in-water emulsion according to example embodiments of the present disclosure.

TABLE 1

| Material | mg/capsule | Percentage |
|---|---|---|
| Choline Chloride | 400 | 46.05% |
| Algarithum ® DHA 530 | 198 | 22.79% |
| Water | 160 | 18.42% |
| Gum Arabic 3020 | 105 | 12.09% |
| 200F Silica | 5 | 0.58% |
| Gellan Gum (low acyl) | 0.65 | 0.07% |

The emulsion as provided in Table 1 has a water activity of about 0.443 and a viscosity of about 1453 cP at room temperature. Viscosity was measured using a rheometer, which provides the viscosity of a solution or emulsion in Pascals per second at a velocity in radians per second. The Pascals per second was then converted to centipoise and the radians per second are converted to RPM to give a final reading.

The emulsion as provided in Table 1 was formulated according to the following steps (1) add water and choline chloride and heat to 80° C., while stirring (2) add gellan gum and shear (3) heat to 80° C. and hold for about 10 minutes (4) cool to room temperature (5) add gum Arabic and DHA oil (6) shear until homogenous (7) add silica (8) stir for 12-18 hours until all visible granules have dissolved.

Table 2 illustrated below, provides an example embodiment of the profile of an oil-in-water emulsion according to example embodiments of the present disclosure.

TABLE 2

| Material | mg/capsule | Percentage |
|---|---|---|
| Choline Chloride | 400 | 47.13% |
| Algarithum ® DHA 530 | 198 | 23.33% |
| Water | 140 | 16.49% |
| Gum Arabic 3020 | 105 | 12.37% |
| 200F Silica | 5 | 0.59% |
| Gellan Gum (low acyl) | 0.65 | 0.077% |

The emulsion provided in Table 2 has a water activity of about 0.463.

The emulsion provided by Table 2 was formulated according to the following steps (1) add water and gellan gum and shear (2) heat to 80° C. for 10 minutes (3) add choline and stir until homogenous (4) cool to room temperature (5) add gum Arabic and then DHA 530 (6) shear until homogenous (7) add silica and stir until silica particles are no longer observed.

Table 3 illustrated below, provides an example embodiment of the profile of an oil-in-water emulsion according to example embodiments of the present disclosure.

TABLE 3

| Material | mg/capsule | Percentage |
|---|---|---|
| Choline Chloride | 400 | 47.13% |
| Algarithum ® DHA 530 | 198 | 23.33% |
| Water | 140 | 16.49% |
| Gum Arabic (A-2010) | 105 | 12.37% |
| 200F Silica | 5 | 0.59% |
| Gellan Gum (low acyl) | 0.65 | 0.077% |

The emulsion provided in Table 3 has a water activity of about 0.384.

The emulsion provided by Table 3 was formulated according to the following steps (1) add choline chloride, water and heat to 80° C. until solubilized (2) add gellan gum (3) shear and heat to 75° C. (4) cool to room temperature and add gum arabic (5) add DHA 530 (6) shear until homogenous (7) add silica and stir for about 3 hours.

Table 4, illustrated below, provides an example embodiment of the profile of an encapsulated oil-in-water emulsion according to example embodiments of the present disclosure.

TABLE 4

| Material | mg/capsule |
|---|---|
| Choline Chloride | 400 |
| Algal Oil AlphaMega 530 | 198 |
| Blend | |
| Gum Acacia | 110 |
| Rosemary extract 12% | 1.00 |
| carnosic acid | |
| Silicon Dioxide Aerosil | 5 |
| 200F | |
| Water, USP | 160 |
| Hypromellose | 188 |

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

1. A oil-in-water emulsion, comprising:
water;
at least one hygroscopic ingredient;
at least one edible oil; and
at least one functional gum;
wherein the oil-in-water emulsion comprise a water activity of less than about 0.6.

2. The emulsion of claim 1, wherein the hygroscopic ingredient further comprises at least one ingredient is selected from the group consisting of choline, carnitine, betaine, L-tyrosine, L-cysteine HCl, L-taurine, L-glutamic acid, N-acetyl-L-tyrosine, alpha ketoglutaric acid, L-arginine HCl, I-ornithine HCl, L-glutamine, L-alanine, L-proline, L-alpha glycerylphosphorylcholine, sorbitol, mannitol, maltitol, and xylitol.

3. The emulsion of claim 2, wherein the hygroscopic ingredient comprises choline.

4. The emulsion of claim 1, wherein the edible oil is selected from the group consisting of any algal oil, hemp oils or extracts, edible seed oils such as sunflower seed oil, tomato seed oil, safflower seed oil, raspberry seed oil, flax seed oil, borage seed oil, fish oils, ahiflower oil, grapeseed oil, sea buckthorn oil, evening primrose oil, argan oil, castor oil, black cumin oil, rice bran oil, ylang ylang oil, oil of oregano, perilla seed oil, wheat germ oil, black currant oil, chia seed oil, inca inchi seed oil, extra virgin olive oil, palm oil, soybean oil, medium chain triglyceride (MCT) oil, and coconut oil.

5. The emulsion of claim 4, wherein the edible oil comprises algal oil, wherein the algal oil comprises docosahexaenoic acid.

6. The emulsion of claim 1, wherein the hygroscopic ingredient comprise from about 30% to about 60% by weight of the emulsion.

7. The emulsion of claim 6, wherein the hygroscopic ingredient comprises from about 40% to about 50% by weight of the emulsion.

8. The emulsion of claim 1, wherein the edible oil comprise from about 10% to about 40% by weight of the emulsion.

9. The emulsion of claim 1, wherein the functional gum comprises gum arabic.

10. The emulsion of claim 9, wherein the functional gum is present in an amount of from about 11% to about 30% by weight.

11. The emulsion of claim 1, wherein the emulsion contains at least one stabilizer, wherein the stabilizer is selected from the group consisting of gellan gum, xantham gum, silica, and combinations thereof.

12. The emulsion of claim 11, wherein the at least one stabilizer is present in an amount of from about 0.25% to about 5% by weight.

13. The emulsion of claim 1, further comprise at least one fat-soluble ingredient, wherein the at least one fat-soluble ingredient is selected from the group consisting of retinol, vitamin E, beta carotene, ubiquinone, lecithin, sunflower lecithin, vitamin D, cannabinoids, hemp extracts, vitamin K, phosphatidyl choline, and combinations thereof.

14. The emulsion of claim 13, wherein the at least one fat-soluble ingredient is present in an amount of less than 50% by weight.

15. The emulsion of claim 1, further comprising at least one water-soluble ingredient, wherein the water soluble ingredient is selected from the group consisting of ascorbic acid, gamma-aminobutyric acid (GABA), sodium chloride, thiamine hydrochloride, riboflavin-5-phosphate sodium, nicotinic acid, folic acid, cyanocobalamin, methylcobalamin, biotin, sodium ascorbate, citicoline, caffeine anhydrous, yohimbine HCl, nicotinamide adenine dinucleotide (NADH), pyridoxine HCl, and combinations thereof.

16. The emulsion of claim 15, wherein the at least one water-soluble ingredient is present in an amount of less than 50% by weight.

17. An oral dosage form, comprising:
a capsule containing the emulsion as provided in claim 1.

18. The oral dosage form of claim 17, wherein the capsule comprises an HPMC-based capsule.

19. The oral dosage form of claim 18, wherein the HPMC-based capsule further comprises gellan gum.

20. A method of improving the health or wellness of a subject, via providing the oral dosage form of claim 17 to the subject.

21. The method of claim 20, wherein the subject is a human.

22. A nutritional supplement, comprising the emulsion as provided in claim 1.
